(19) United States
(12) Patent Application Publication
(10) Pub. No.: US 2021/0350721 A1
(43) Pub. Date: Nov. 11, 2021

(54) Nutritional Aid

(71) Applicant: Tony DiFruscio, St. Catharines (CA)

(72) Inventor: Tony DiFruscio, St. Catharines (CA)

(21) Appl. No.: 17/383,163

(22) Filed: Jul. 22, 2021

Related U.S. Application Data
(63) Continuation of application No. 16/179,657, filed on Nov. 2, 2018, now abandoned.
(60) Provisional application No. 62/580,484, filed on Nov. 2, 2017.

(51) Int. Cl. G09B 19/00; G16H 20/60

(52) U.S. Cl. CPC G09B 19/0092; G16H 20/60

(57) ABSTRACT

Instructions executable by a computer form one aspect of the invention. The instructions, when executed by the computer, provide a computing facility adapted to: (i) allow a user to select one or more components of a meal; (ii) carry out a calculation to produce a meal, built from the selected components, that meets a caloric goal of the user; and (iii) display the results of the calculation expressed, in respect of each of the selected components, as a portion of a serving or a size of a serving.

### CROSS-REFERENCE TO RELATED APPLICATION

This application is a continuation application based on U.S. patent application Ser. No. 16/179,657 filed Nov. 2, 2018 which claims the benefit of U.S. Provisional Patent Application Ser. No. 62/580,484 filed Nov. 2, 2017.

### BACKGROUND OF THE INVENTION

### 1. Field of the Invention

The invention relates to the field of nutrition.

### 2. Prior Art

Weight loss apps are well known. It is typical for apps to give users tools to count calories for the day.

### SUMMARY OF THE INVENTION

Instructions executable by a computer form one aspect of the invention. The instructions, when executed by the computer, provide a computing facility adapted to: (i) allow a user to select one or more components of a meal; (ii) carry out a calculation to produce a meal, built from the selected components, that meets a caloric goal of the user; and (iii) display the results of the calculation expressed, in respect of each of the selected components, as a portion of a serving or a size of a serving.

According to another aspect, the functionality can be adapted to allow a user to fix the amount of one or more components of the meal.

According to another aspect, the functionality can be further adapted to produce the caloric goal of the user based upon user characteristics and preferences.

According to another aspect, the functionality can be further adapted to collect the user characteristics based upon user inputs including age, sex, weight and desired weight loss.

According to another aspect, the calculation can produce the meal based upon a caloric goal for the meal, the caloric goal for the meal itself being based upon a caloric goal for the day and user preferences respecting the distribution of calories for each meal of the day.

### BRIEF DESCRIPTION OF THE DRAWINGS

FIG. 1 is a screen capture of the personal info screen of the app;

FIG. 2 is a screen capture of the weight/goal screen of the app;

FIG. 3 is a screen capture of the activity screen of the app;

FIG. 4 is a screen capture of the meals screen of the app;

FIG. 5 is a screen capture of the macro nutrition screen of the app;

FIG. 6 is a screen capture of the home page of the app;

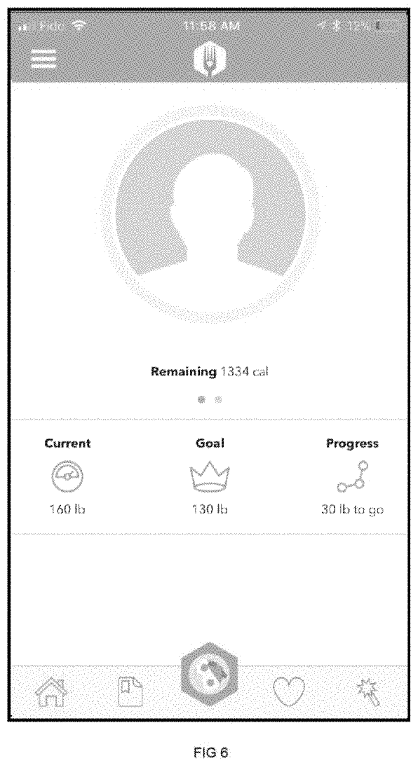

FIG. 7 is a screen capture of the selection screen of the app;

FIG. 8 is a screen capture of the search field screen of the app;

FIG. 9 is a screen capture of the meal screen of the app; and

FIG. 10 is a screen capture of the override screen of the app.

### DETAILED DESCRIPTION OF THE PREFERRED EMBODIMENTS

The exemplary app has two main modules

### Set Up

In the set-module, the app collects data about the user and his or her goals.

Specifically, the app collects:

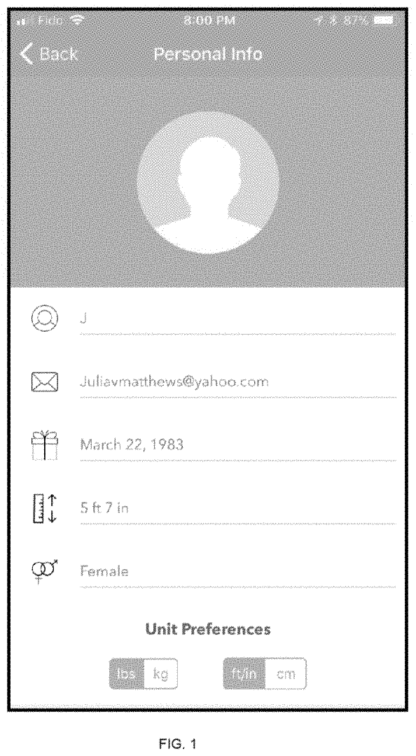

as shown in FIG. 1
User Name
User Height
User Sex
User Birthdate

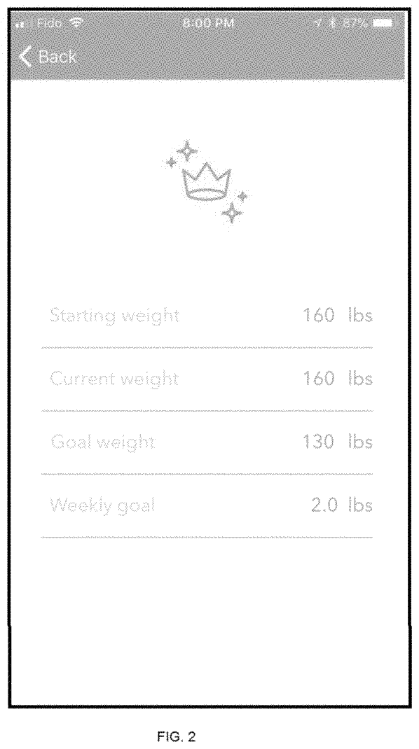

as shown in FIG. 2
Starting Weight
Current weight
Goal Weight
Weekly weight loss goal

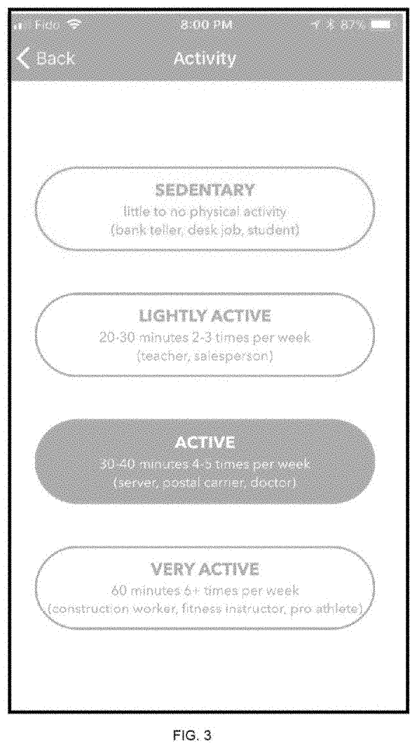

as shown in FIG. 3
Activity level

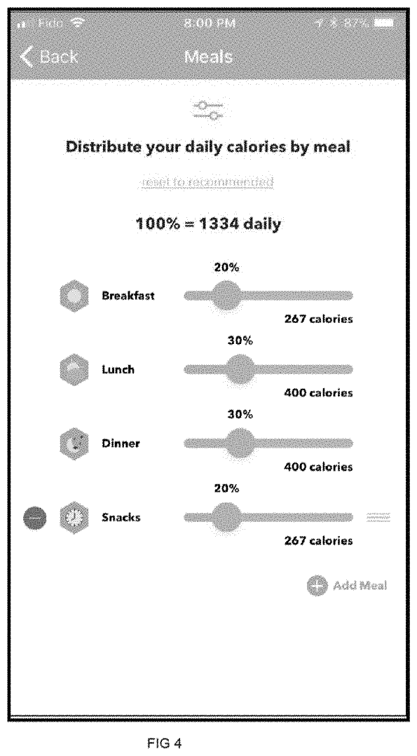

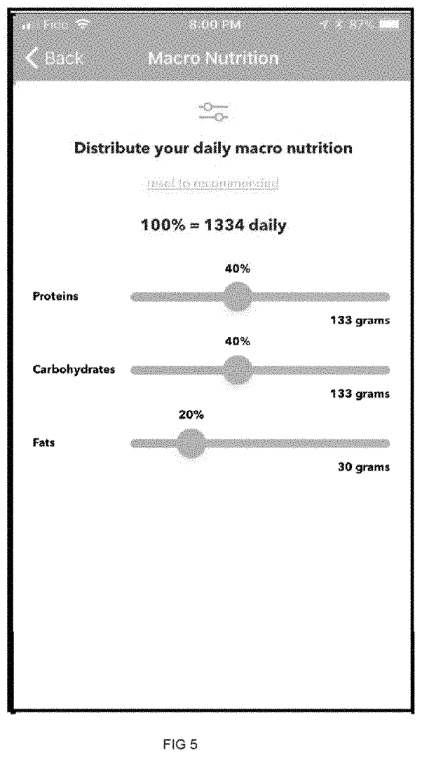

as shown in FIG. 4
Desired daily calorie distribution, by meal and, as shown in FIG. 5
Desired mix of protein, carbohydrate and fat
   The user can enter a current weight at any time in the page of FIG. 2.

Based upon the above, the system generates a daily calorie target based upon calorie calculation methodology that is well known to persons of ordinary skill in the art and accordingly is neither required nor disclosed herein.

### Meal Selection

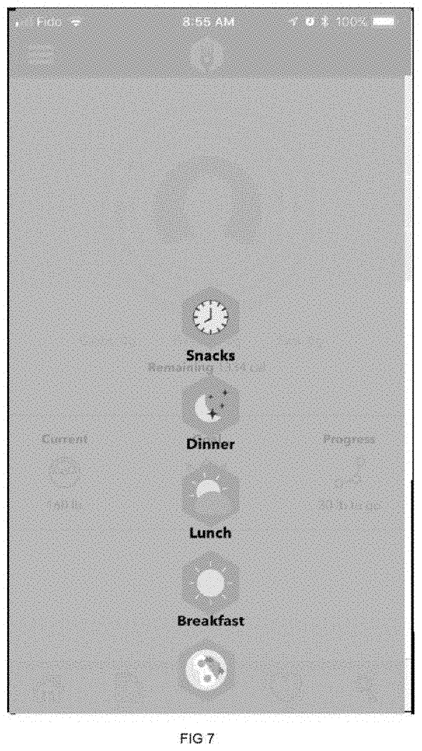

Meal selection starts by selecting the bacon-and-eggs icon at the base of the home page, which triggers display of a meal selector page, shown in FIG. 7

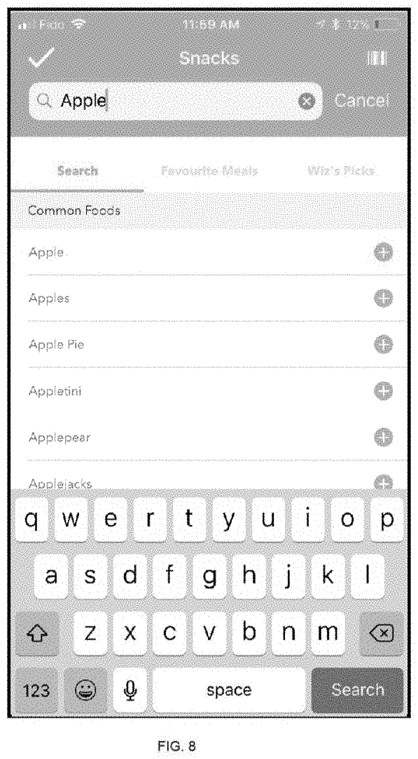

The user then selects a meal to be eaten [breakfast, lunch, dinner or snack] from the selection screen and then selects components proposed to be eaten at the meal based upon favourites and a search field, as indicated in FIG. 8

As each component is selected, it is added to a list.

At any time, a serving amount can be locked or manually edited.

The serving amount shown in respect of each component varies.

By way of background, databases that show standard serving sizes of various foodstuffs, as well as associated caloric and other nutritional information are well known.

When a meal entry commences, as a component is added, it is typical that the standard serving size for that component will not exceed the target calories for the meal; accordingly, as the first component is added, a full serving will be shown.

As additional components are added to the meal, the serving sizes shown reduce, otherwise than in respect of locked components, such that, where possible, the target calories for the meal are not exceeded.

The serving sizes are calculated based upon an iterative process that relies upon the database of components, with associated standard serving sizes and calories, as is well-known, along with a predefined minimum increment for each food serving unit that is easily measured by a user in practise.

Minimum Increments that have been Found Useful:

Serving

| | |
|---|---|
| Whole number, i.e. | .25 |
| 1 chicken breast | |
| Grams | 5 |
| Ounces | .2 |
| Mls | 5 |
| Tsp | ¼ |
| Tbsp | ¼ |
| cups | ¼ |

By way of further explanation, the iterative process commences with a division of the target calories available for distribution (net of locked calories) amongst the unlocked components, which normally results in irregular serving sizes, i.e. 0.87756 of a chicken breast.

The serving size of each unlocked component is automatically reduced therefrom to the nearest multiple of the minimum serving size, i.e. 0.87756 chicken breast is stepped down to 0.75 chicken breast [3×minimum service size of ¼].

Thereafter, the unlocked foods are sequentially looped through, and minimum increments thereof are added to the serving size if such addition does not cause the total calories to exceed the target calories.

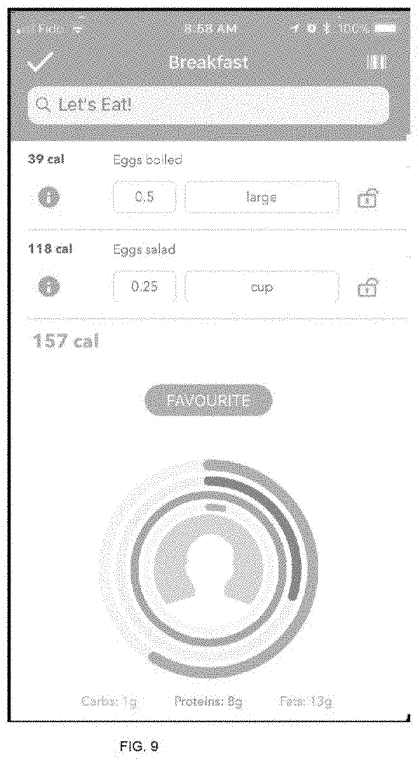

If the loop results in a change, the loop is repeated; if no change is made in a loop, the looping terminates. This results in a meal that is close to the target calories using practical increments for the selected units, as shown in FIG. 9.

The system displays, as a proportion of the target of the day, the amounts of carbohydrates, proteins and fats represents by the meals accounted for in the day, to allow the user to modify and lock components of the meal, to adjust the nutritional division appropriately. Changes cause the above iterative process to be repeated.

The user may modify and lock the serving size of any component of the meal, by entering an amount other than the suggested serving size; a user can input that which is actually eaten.

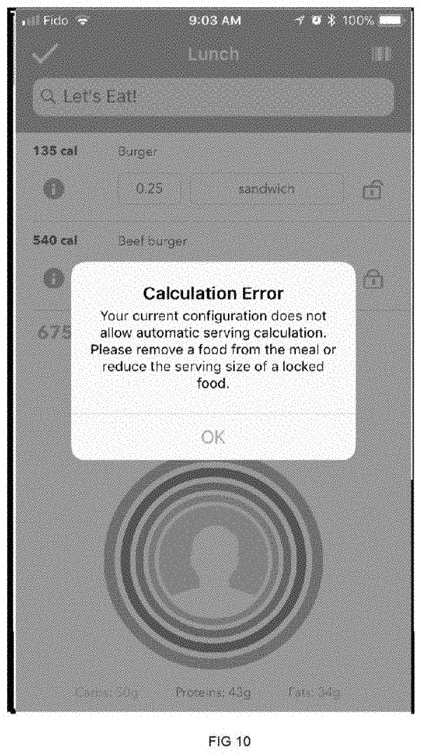

When a user exceeds the target calories for that meal, a notification/alert is triggered; by hitting “OK”, the user can exceed the target calories, as indicated in FIG. 10

### VARIATIONS

Whereas a single embodiment of the app is shown and described, variations are possible.

For example, similar iterative techniques could be utilized to tailor the amounts of the components of the meal to maximize, for example, protein, or to ensure that a target amount of, for example, sodium, is not exceeded.

Accordingly, the invention should be understood to be limited only by the accompanying claims, purposively construed.

What is claimed is:

1. Instructions executable by a computer, the instructions, when executed by the computer, providing a computing facility adapted to:
allow a user to select one or more components of a meal;
carry out a calculation to produce a meal, built from the selected components, that meets a caloric goal of the user; and
display the results of the calculation expressed, in respect of each of the selected components, as a portion of a serving or a size of a serving.

2. Instructions according to claim 1, wherein the functionality is adapted to allow a user to fix the amount of one or more components of the meal, provided that such fixed amount allows for the caloric goal to be met.

3. Instructions according to claim 1, wherein the functionality is further adapted to produce the caloric goal of the user based upon user characteristics and preferences.

4. Instructions according to claim 4, wherein the functionality is further adapted to collect the user characteristics based upon user inputs including age, sex, weight and desired weight loss.

5. Instructions according to claim 1, wherein the calculation produces the meal based upon a caloric goal for the meal, the caloric goal for the meal itself being based upon a caloric goal for the day and user preferences respecting the distribution of calories for each meal of the day.

6. Instructions according to claim 1, wherein the functionality is adapted to:
collect user targets respecting the proportion of caloric intake represented by carbohydrates, protein and fat and collects; and
display, as a proportion of the target for the day, the amounts of carbohydrates, proteins and fats represented by the meals accounted for in the day.
